(19) United States
(12) Patent Application Publication
(10) Pub. No.: US 2022/0023029 A1
(43) Pub. Date: Jan. 27, 2022

(54) PROSTHESIS TO REPLACE A FLEXOR TENDON PULLEY

(71) Applicants: Ali Moradi, Mashhad (IR); Mahla Daliri, Mashhad (IR); Seyedali Mousavi Shayegh, Mashhad (IR); Mohammadhossein Ebrahimzadeh, Mashhad (IR)

(72) Inventors: Ali Moradi, Mashhad (IR); Mahla Daliri, Mashhad (IR); Seyedali Mousavi Shayegh, Mashhad (IR); Mohammadhossein Ebrahimzadeh, Mashhad (IR)

(73) Assignees: Ali Moradi, Mashhad (IR); Seyedali Mousavi Shayegh, Mashhad (IR); Mohammadhossein Ebrahimzadeh, Mashhad (IR)

(21) Appl. No.: 17/495,714

(22) Filed: Oct. 6, 2021

Related U.S. Application Data
(60) Provisional application No. 63/087,889, filed on Oct. 6, 2020.

(51) Int. Cl. A61F 2/08; A61L 27/12; A61L 27/20; A61L 27/06; A61L 27/26

(52) U.S. Cl. CPC A61F 2/0811; A61L 27/12; A61L 27/20; A61L 27/06; A61L 2430/10; A61F 2230/0069; A61F 2002/0864; A61F 2002/0817; A61L 27/26

(57) ABSTRACT

A prosthesis to replace a flexor tendon pulley. The prosthesis comprises a tendon holding member with a hollow cylindrical shape and a connecting plate attached to the tendon holding member. The tendon holding member is configured to receive and hold a tendon of the human hand. The connecting plate is configured to be attached to a phalange of the human hand. The tendon holding member comprises an outer layer on an outer surface of the tendon holding member and an inner layer on an inner surface of the tendon holding member. The outer layer comprises hydroxyapatite to secure the tendon holding member to the surrounding tissues. The inner layer comprises of hyaluronic acid to secure the tendon holding member to the surrounding tissues.

### CROSS-REFERENCE TO RELATED APPLICATION

This application claims the benefit of priority from pending U.S. Provisional Patent Application Ser. No. 63/087,889, filed on Oct. 6, 2020, and entitled “ARTIFICIAL DIGITAL ANNULAR PULLEY” which is incorporated herein by reference in its entirety.

### TECHNICAL FIELD

The present disclosure generally relates to prostheses. This disclosure, more particularly, relates to a prosthesis to replace a tendon sheath in a human body for compensating a damaged tendon pulley system. For example, the prosthesis may replace a flexor tendon pulley of a human hand for compensating an associated damaged tendon pulley system.

### BACKGROUND

Fingers of a human hand are moved by flexor and extensor tendons which extend from corresponding tensor and extensor muscles in the human forearm. Extrinsic flexor tendons, of which there may be two for each finger, are attached to flexor digitorum profundus and flexor digitorum superficialis muscles. At a level of a metacarpophalangeal joint, each superficialis tendon splits longitudinally into two parts. The two parts pass around the profundus tendon and then reunite before separating again to attach to either side of the palmar surfaces of the base of the middle phalanx. This peculiar arrangement of the superficialis tendon provides a tunnel that allows the profundus tendon to become superficial. The effect of the peculiar arrangement, so far as the superficialis tendon is concerned, is to increase the lever arm of the tendon at the proximal interphalangeal joint, thereby enabling a powerful grip of the fingers to be exerted. The tendon of the flexor digitorum profundus muscle eventually inserts into the bone of the palmar surfaces of the distal phalanx.

Tendons of digital flexors are held in proximity to the phalanges of each finger in each case by pulleys which act to prevent so-called “bowstringing” of the tendons and ensure that tendon's pull produces immediate movement at the interphalangeal joint. Damage to these pulleys may occur in several ways. For example, they may be cut, severed, or crushed in an accident. Also, forces applied to related tendons in rock-climbing may rupture the pulleys. This may produce displacement of a vector or line of action of a tendon on a finger, resulting in deformity of the finger and loss of normal movement and power of action.

Sometimes, the damaged pulleys may be reconstructed with nearby tissues. This is, however, not always feasible or desirable. In some cases, reconstruction with a sufferer's own tissues is not satisfactory because they are likely to be weakened by a disease or else they are likely to be weakened subsequently. The reconstructed result is therefore prone to deteriorate over time. There is, therefore, a need for a prosthesis that is able to replace a flexor tendon pulley of a human hand and can withstand abnormal forces exerted upon tendons of the hand and fingers.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes a prosthesis to replace a tendon sheath of a human body. For example, the prosthesis may replace a flexor or extensor tendon pulley. In an exemplary embodiment, the prosthesis may comprise a tendon holding member and a connecting plate.

In an exemplary embodiment, the tendon holding member may comprise a hollow cylindrical shape. In an exemplary embodiment, the tendon holding member may comprise polyurethane, polycaprolactone, or a combination thereof. In an exemplary embodiment, an inner diameter of an inner surface of the tendon holding member may be in a range between 4 mm and 12 mm. In an exemplary embodiment, the inner diameter of the inner surface of the tendon holding member may be 8 mm. In an exemplary embodiment, a length of the tendon holding member may be in a range between 5 mm and 15 mm. In an exemplary embodiment, the length of the tendon holding member may be 10 mm. In an exemplary embodiment, a thickness of the tendon holding member may be in a range between 10 μm and 300 μm. In an exemplary embodiment, the thickness of the tendon holding member may be 200 μm. the tendon holding member configured to receive and hold a tendon of the human hand.

In an exemplary embodiment, the tendon holding member may comprise an outer layer on an outer surface of the tendon holding member and an inner layer on an inner surface of the tendon holding member. In an exemplary embodiment, a thickness of the outer layer may be in a range between 5 μm and 50 μm. In an exemplary embodiment, the thickness of the outer layer may be 12 μm. In an exemplary embodiment, the outer layer may comprise hydroxyapatite. In an exemplary embodiment, the outer layer may be configured to form a fibrous tissue between the outer surface of the tendon holding member and surrounding tissues around the tendon holding member.

In an exemplary embodiment, a thickness of the inner layer may be in a range between 5 μm and 50 μm. In an exemplary embodiment, a thickness of the inner layer may be 12 μm. In an exemplary embodiment, the inner layer may comprise hyaluronic acid. In an exemplary embodiment, the inner layer may be configured to prevent adhesion of the tendon of the human hand to the inner surface of the tendon holding member.

In an exemplary embodiment, the connecting plate may have a rectangular shape. In an exemplary embodiment, the connecting plate may be attached to the tendon holding member. In an exemplary embodiment, a length of the connecting plate may be in a range between 7 mm and 20 mm. in an exemplary embodiment, the length of the connecting plate may coincide a length of the tendon holding member. In an exemplary embodiment, the length of the connecting plate may be 15 mm. In an exemplary embodiment, a width of the connecting plate may be in a range between 4 mm and 12 mm. In an exemplary embodiment, the width of the connecting plate may coincide the inner diameter of the inner surface of the tendon holding member. In an exemplary embodiment, the width of the connecting plate may be 9 mm. In an exemplary embodiment, a thickness of the connecting plate may be in a range between 0.5 mm and 2 mm. In an exemplary embodiment, the thickness of the connecting plate may be 1 mm. In an exemplary embodiment, the connecting plate may comprise titanium. In an exemplary embodiment, the connecting plate may be configured to be attached to a phalange of the human hand. In an exemplary embodiment, the phalange of the human hand may be associated with the tendon of the human hand. In an exemplary embodiment, the connecting plate may comprise a plurality of connecting holes. In an exemplary embodiment, a connecting hole from the plurality of connecting holes may be configured to receive a connecting screw. In an exemplary embodiment, the connecting screw may be configured to be screwed into the phalange of the human hand and, to thereby, secure the connecting plate to the phalange of the human hand.

In an exemplary embodiment, the plurality of connecting holes may comprise a first connecting hole at a first corner of the connecting plate, a second connecting hole at a second corner of the connecting plate, a third connecting hole at a third corner of the connecting plate, a fourth connecting hole at a fourth corner of the connecting plate, and a fifth connecting hole at a center of the connecting plate.

In an exemplary embodiment, the tendon holding member may comprise an access hole on a top section of the tendon holding member. In an exemplary embodiment, the access hole may be aligned with the fifth connecting hole. In an exemplary embodiment, the access hole may be configured to provide access to the fifth connecting hole.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates a perspective view of a prosthesis to replace a flexor tendon pulley of a human hand, consistent with one or more exemplary embodiments of the present disclosure.

FIG. 1B illustrates a top view of a prosthesis, consistent with one or more exemplary embodiments of the present disclosure.

FIG. 1C illustrates a side view of a prosthesis, consistent with one or more exemplary embodiments of the present disclosure.

FIG. 1D illustrates a sectional front view of a prosthesis, consistent with one or more exemplary embodiments of the present disclosure.

FIG. 2A illustrates a tendon holding member, consistent with one or more exemplary embodiments of the present disclosure.

FIG. 2B illustrates a perspective view of a tendon holding member in a scenario in which tendon holding member holds a tendon, consistent with one or more exemplary embodiments of the present disclosure.

FIG. 3A illustrates a perspective view of a connecting plate, consistent with one or more exemplary embodiments of the present disclosure.

FIG. 3B illustrates a top view of a connecting plate, consistent with one or more exemplary embodiments of the present disclosure.

FIG. 3C illustrates a side view of a connecting plate, consistent with one or more exemplary embodiments of the present disclosure.

FIG. 3D illustrates a front view of a connecting plate, consistent with one or more exemplary embodiments of the present disclosure.

FIG. 3E illustrates a bottom view of a connecting plate, consistent with one or more exemplary embodiments of the present disclosure.

FIG. 3F shows a prosthesis in a scenario in which the prosthesis is attached to a phalange of a human hand, consistent with one or more exemplary embodiment of the present disclosure.

FIG. 4 illustrates a front view of a tendon holding member, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

The present disclosure is directed to exemplary embodiments of a prosthesis to replace a tendon sheath of a human body. For example, the prosthesis may replace a flexor or extensor tendon pulley. An exemplary prosthesis comprises a tendon holding member and a connecting plate. In an exemplary embodiment, the connecting plate may be attached to a bottom section of the tendon holding member which has a hollow cylindrical shape. A surgeon may attach the connecting plate to a phalange of a human hand and pass a flexor tendon through the tendon holding member. An exemplary prosthesis may replace a flexor tendon pulley and may act as the flexor tendon pulley for the flexor tendon. In an exemplary embodiment, a tendon holding member may comprise of an outer layer, comprising hydroxyapatite, on an outer surface of the tendon holding member that may help an exemplary prosthesis to be secured to surrounding tissues around the prosthesis. In an exemplary embodiment, the tendon holding member may also comprise of an inner layer, comprising hyaluronic acid, on an inner surface of the tendon holding member that may prevent the adhesion of the tendon to the inner surface of the tendon holding member and, to thereby, provide a facility for the tendon to move and slide easily inside the tendon holding member. The prosthesis may replace any of pulleys or tendon sheaths in human body. For example, the prosthesis may replace any of pulleys of a human hand (A1-A5).

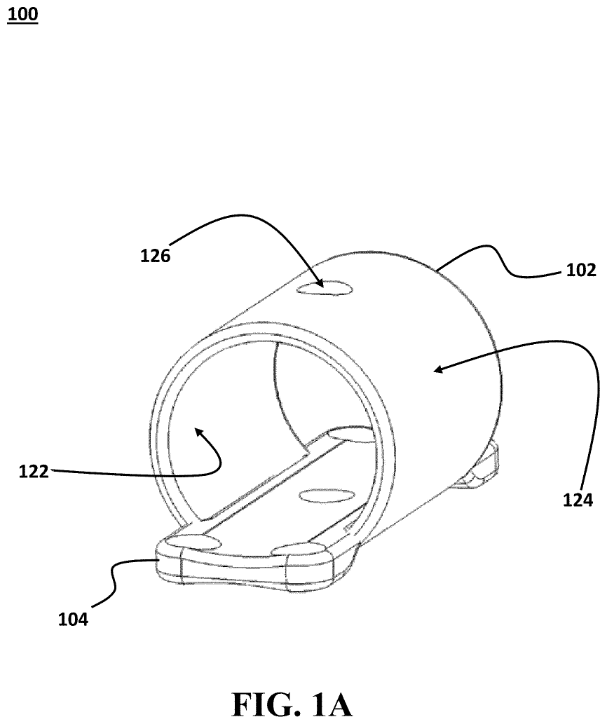

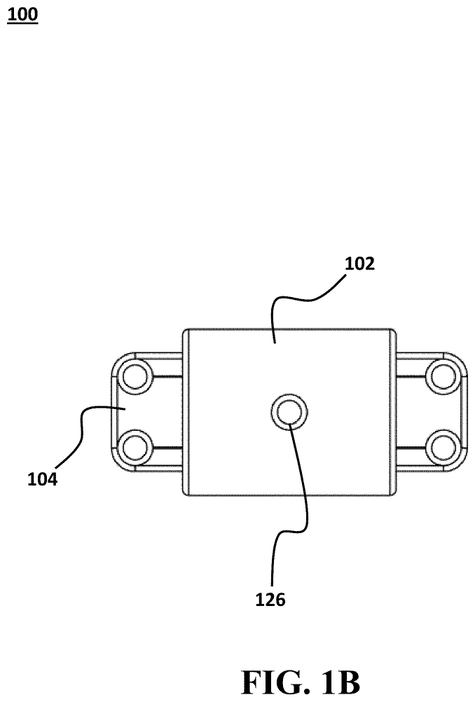

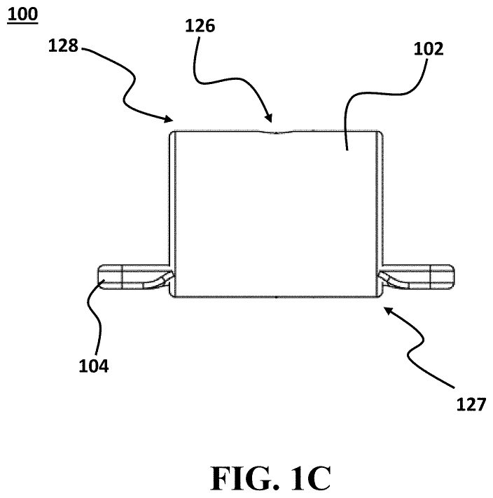

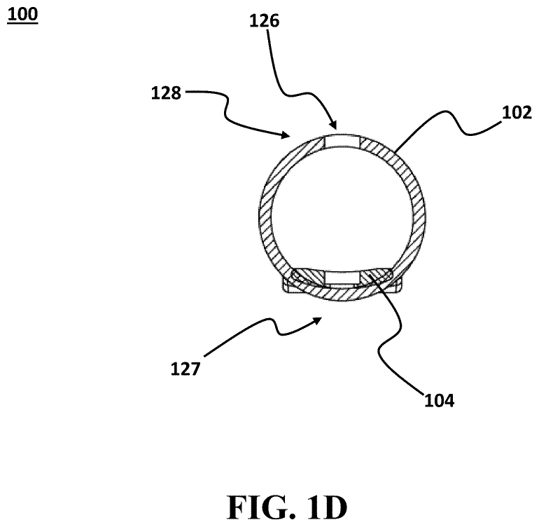

FIG. 1A shows a perspective view of a prosthesis 100 to replace a flexor tendon pulley of a human hand, consistent with one or more exemplary embodiments of the present disclosure. FIG. 1B shows a top view of prosthesis 100, consistent with one or more exemplary embodiments of the present disclosure. FIG. 1C shows a side view of prosthesis 100, consistent with one or more exemplary embodiments of the present disclosure. FIG. 1D shows a sectional front view of prosthesis 100, consistent with one or more exemplary embodiments of the present disclosure. As shown in FIGS. 1A, 1B, 1C, and 1D, in an exemplary embodiment, prosthesis 100 may comprise a tendon holding member 102 and a connecting plate 104.

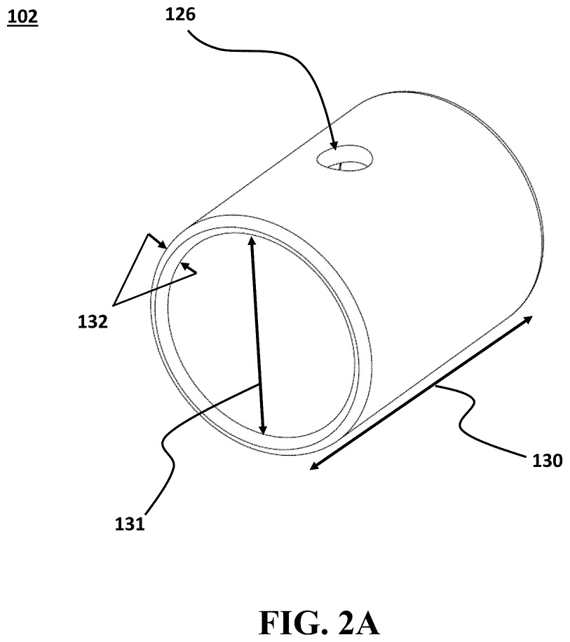

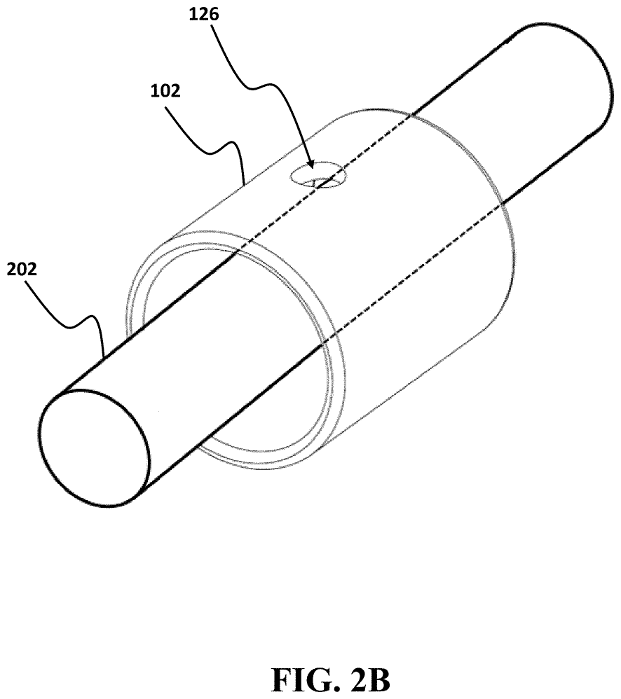

FIG. 2A shows tendon holding member 102, consistent with one or more exemplary embodiments of the present disclosure. As shown in FIG. 2A, in an exemplary embodiment, tendon holding member 102 may have a hollow cylindrical shape. In an exemplary embodiment, tendon holding member 102 may have any other hollow structures such as a hollow rectangular structure and a hollow triangular structure. In an exemplary embodiment, tendon holding member 102 may be configured to receive and hold a tendon of the human hand. In an exemplary embodiment, when tendon holding member 102 holds a tendon, it may mean that the tendon is placed inside tendon holding member 102. In an exemplary embodiment, when tendon holding member 102 holds a tendon, the tendon may be able to move and slide easily inside tendon holding member 102 without adhering to surrounding tissues. In an exemplary embodiment, tendon holding member 102 may comprise polyurethane, polycaprolactone, or a combination thereof. In an exemplary embodiment, a length 130 of tendon holding member 102 may be in a range between 5 mm and 15 mm. In an exemplary embodiment, length 130 of tendon holding member 102 may refer to an edge of the hollow cylindrical shape of tendon holding member 102. In an exemplary embodiment, length 130 of tendon holding member 102 may be 10 mm. In an exemplary embodiment, an inner diameter 131 of an inner surface of tendon holding member 102 may be in a range between 4 mm and 12 mm. In an exemplary embodiment, inner diameter 131 of the inner surface of tendon holding member 102 may be 8 mm. In an exemplary embodiment, a thickness 132 of tendon holding member 102 may be in a range between 10 μm and 300 μm. In an exemplary embodiment, thickness 132 of tendon holding member 102 may refer to a smallest distance between an inner surface of tendon holding member 102 and an outer surface of tendon holding member 102. In an exemplary embodiment, thickness 132 of tendon holding member 102 may be 200 μm. FIG. 2B shows a perspective view of tendon holding member 102 in a scenario in which tendon holding member 102 holds a tendon 202, consistent with one or more exemplary embodiments of the present disclosure. In an exemplary embodiment, tendon 202 may refer to any tendon in a human body. For example, tendon 202 may refer to a flexor or extensor tendon in a human hand. In an exemplary embodiment, the hollow structure of tendon holding member 102 may provide a space through which tendon 202 may pass. In an exemplary embodiment, when tendon holding member 102 receives and holds tendon 202, it may mean that tendon 202 is disposed inside the hollow structure of tendon holding member 102 as shown in FIG. 2B.

As shown in FIGS. 1A, 1B, 1C, and 1D, in an exemplary embodiment, connecting plate 104 may be attached to tendon holding member 102. In an exemplary embodiment, connecting plate 104 may comprise titanium. In an exemplary embodiment, connecting plate 104 may be disposed inside tendon holding member 102. In an exemplary embodiment, connecting plate 104 may be attached to an inner surface 122 of tendon holding member 102. In an exemplary embodiment, connecting plate 104 may be attached to a bottom section 127 of tendon holding member 102. In an exemplary embodiment, bottom section 127 of tendon holding member 102 may refer to a section of tendon holding member 102 that may be configured to be directly in contact with an exemplary phalange of a human hand. In an exemplary embodiment, connecting plate 104 may be fixedly attached to inner surface 122 of tendon holding member 102. In an exemplary embodiment, when connecting plate 104 is fixedly attached to inner surface 122 of tendon holding member 102, it may mean that connecting plate 104 is attached to inner surface 122 of tendon holding member 102 in such a way that any relative movement between connecting plate 104 and tendon holding member 102 is prevented. In an exemplary embodiment, connecting plate 104 and tendon holding member 102 may be manufactured seamlessly to create an integrated and/or unitary part. In an exemplary embodiment, connecting plate 104 may be detachably attached to inner surface 122 of tendon holding member 102. In an exemplary embodiment, connecting plate 104 may be attached to inner surface 122 of tendon holding member 102 by utilizing a screw. In an exemplary embodiment, by tightening the screw between connecting plate 104 and inner surface 122 of tendon holding member 102, connecting plate 104 may be attached to inner surface 122 of tendon holding member 102 and by loosening the screw, connecting plate 104 may be detached to inner surface 122 of tendon holding member 102. In an exemplary embodiment, when connecting plate 104 is detachably attached to inner surface 122 of tendon holding member 102, it may mean that a user (for example a surgeon) may easily attach connecting plate 104 to inner surface 122 of tendon holding member 102 and/or detach connecting plate 104 from inner surface 122 of tendon holding member 102.

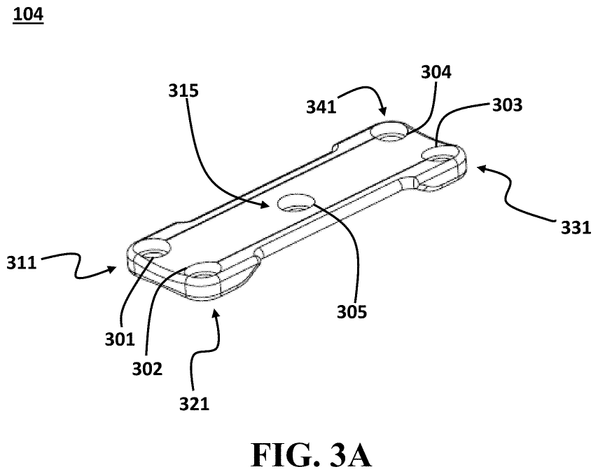

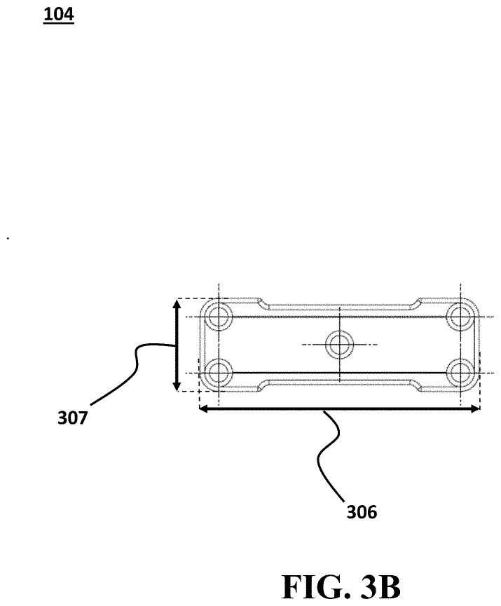

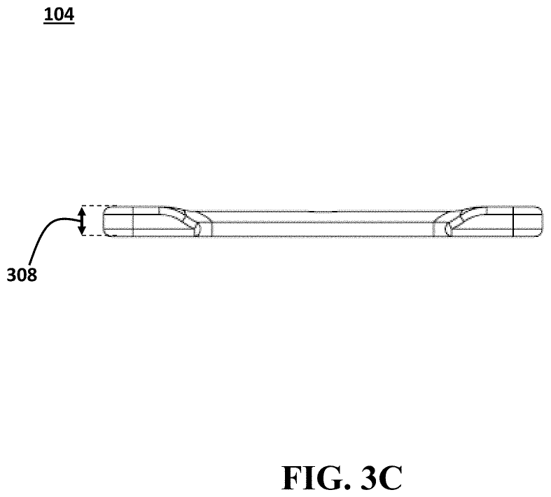

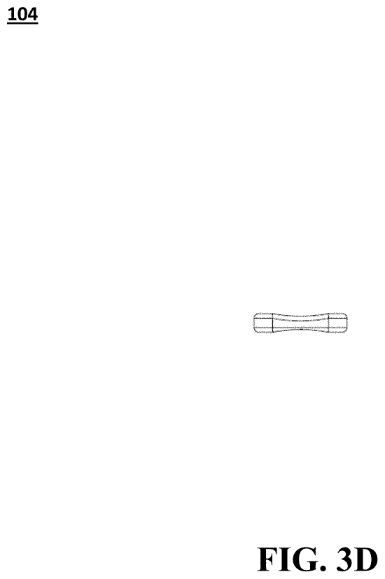

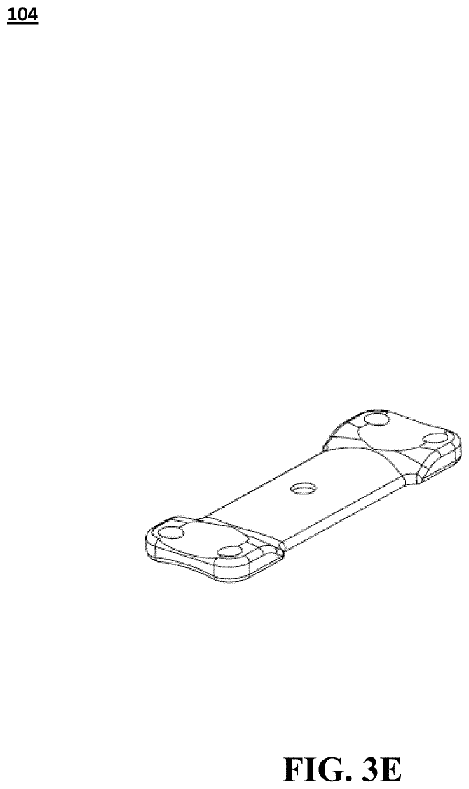

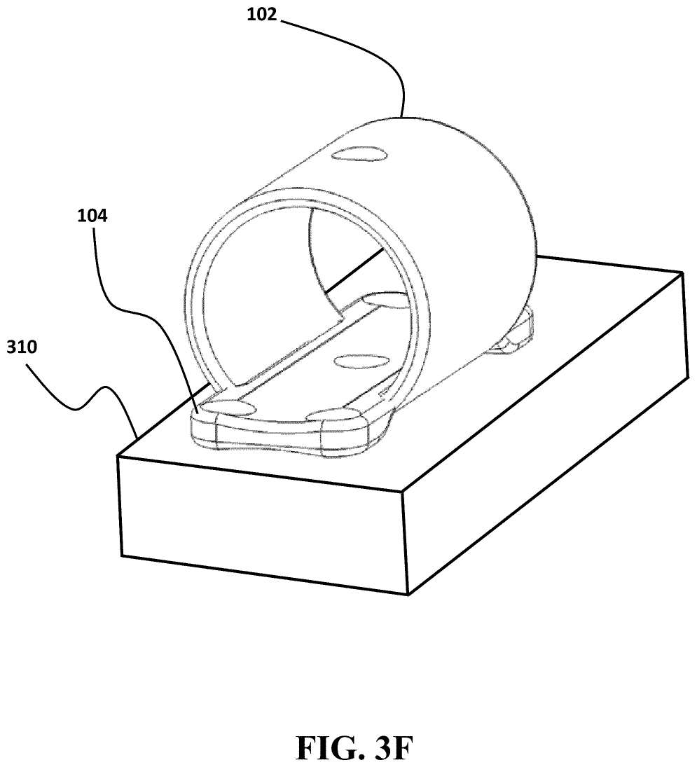

FIG. 3A shows a perspective view of connecting plate 104, consistent with one or more exemplary embodiments of the present disclosure. FIG. 3B shows a top view of connecting plate 104, consistent with one or more exemplary embodiments of the present disclosure. FIG. 3C shows a side view of connecting plate 104, consistent with one or more exemplary embodiments of the present disclosure. FIG. 3D shows a front view of connecting plate 104, consistent with one or more exemplary embodiments of the present disclosure. FIG. 3E shows a bottom view of connecting plate 104, consistent with one or more exemplary embodiments of the present disclosure. In an exemplary embodiment, connecting plate 104 may be configured to be attached to a phalange of a human hand. In an exemplary embodiment, a “phalange” may refer to a bone of a finger of a human hand. In an exemplary embodiment, a surgeon may attach connecting plate 104 to a phalange of a human hand. FIG. 3F shows prosthesis 100 in a scenario in which connecting plate 104 is attached to a phalange 310 of a human hand, consistent with one or more exemplary embodiment of the present disclosure. It is noteworthy that phalange 310, which is shown in FIG. 3F, is a schematic representation of an exemplary phalange in a human body but for all phalanges in a human body, regardless of the shape of the phalange, connecting plate 104 may be attached to phalange 310 in a similar way.

In an exemplary embodiment, connecting plate 104 may have a rectangular shape. In an exemplary embodiment, a length 306 of connecting plate 104 may be in a range between 7 mm and 20 mm. In an exemplary embodiment, length 306 of connecting plate 104 may be 15 mm. In an exemplary embodiment, a width 307 of connecting plate 104 may be in a range between 4 mm and 12 mm. In an exemplary embodiment, width 307 of connecting plate 104 may be 9 mm. In an exemplary embodiment, a thickness 308 of connecting plate 104 may be in a range between 0.5 mm and 2 mm. In an exemplary embodiment, thickness 308 of connecting plate 104 may be 1 mm. In an exemplary embodiment, connecting plate 104 may have any other shapes such as triangular or circular. In an exemplary embodiment, connecting plate 104 may comprise a plurality of connecting holes. In an exemplary embodiment, the plurality of connecting holes may comprise a first connecting hole 301, a second connecting hole 302, a third connecting hole 303, a fourth connecting hole 304, and a fifth connecting hole 305. In an exemplary embodiment, first connecting hole 301 may be located at a first corner 311 of connecting plate 104. In an exemplary embodiment, second connecting hole 302 may be located at a second corner 312 of connecting plate 104. In an exemplary embodiment, third connecting hole 303 may be located at a third corner 313 of connecting plate 104. In an exemplary embodiment, fourth connecting hole 304 may be located at a fourth corner 314 of connecting plate 104. In an exemplary embodiment, fifth connecting hole 305 may be located at a center 315 of connecting plate 104. In an exemplary embodiment, the arrangement of the plurality of connecting holes may help connecting plate 104 to attach to phalange 310 of a human hand firmly and does not loose over time.

In an exemplary embodiment, each connecting hole from the plurality of connecting holes may be configured to receive a connecting screw. In an exemplary embodiment, a surgeon may insert a connecting screw to a connecting hole from the plurality of connecting holes and then may screw the connecting screw into phalange 310 so that connecting plate 104 is secured to phalange 310. In an exemplary embodiment, a surgeon may attach and fix prosthesis 100 to phalange 310 of a human hand and then pass tendon 202 through tendon holding member 102 so that prosthesis 100 may act as a flexor tendon pulley for tendon 202. In an exemplary embodiment, the surgeon may attach and fix prosthesis 100 to a part of phalange 310, where the flexor tendon pulley had previously been there.

In an exemplary embodiment, tendon holding member 102 may comprise an access hole 126 on a top section 128 of tendon holding member 102. In an exemplary embodiment, top section 128 of tendon holding member 102 may refer to a section of tendon holding member 102 which may be located opposite to the bottom section 127 of tendon holding member 102. In an exemplary embodiment, access hole 126 may be aligned with fifth connecting hole 305. In an exemplary embodiment, fifth connecting hole 305 may be configured to provide access to fifth connecting hole 305. In an exemplary embodiment, a surgeon may pass a connecting screw through access hole 126 and insert the connecting screw into fifth connecting hole 305. In an exemplary embodiment, access hole 126 may provide a facility for a surgeon that allows the surgeon to use a screwdriver to insert a connecting screw into the fifth connecting hole and screw the connecting screw to phalange 310 of a human hand.

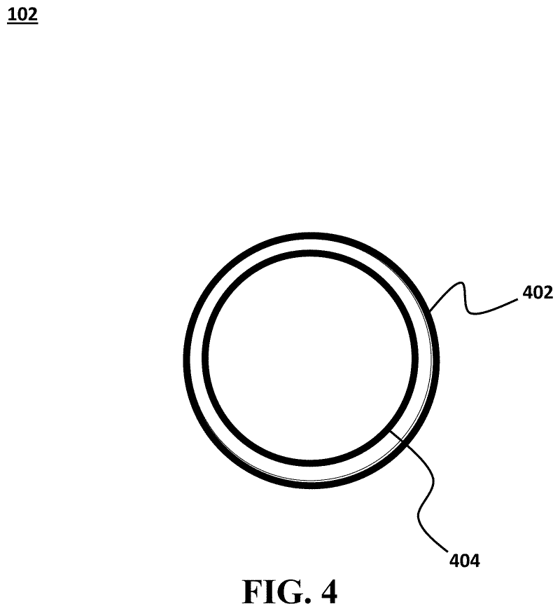

FIG. 4 shows a front view of tendon holding member 102, consistent with one or more exemplary embodiments of the present disclosure. As shown in FIG. 4, in an exemplary embodiment, tendon holding member 102 may comprise an outer layer 402 on an outer surface 124 of tendon holding member 102. In an exemplary embodiment, outer layer 402 may comprise hydroxyapatite. In an exemplary embodiment, outer layer 402 may help in forming a fibrous tissue between outer surface 124 of tendon holding member 102 and surrounding tissues around tendon holding member 102 and, to thereby, may help securing prosthesis 100 to surrounding tissues around prosthesis 100. In an exemplary embodiment, the hydroxyapatite present in outer layer 402 may aid in forming the fibrous tissue between outer surface 124 of tendon holding member 102 and surrounding tissues around tendon holding member 102. In an exemplary embodiment, a thickness of outer layer 402 may be in a range between 5 μm and 50 μm. In an exemplary embodiment, the thickness of outer layer 402 may be 12 μm. In an exemplary embodiment, outer layer 402 may be coated onto outer surface 124 of tendon holding member 102.

As further shown in FIG. 4, in an exemplary embodiment, tendon holding member 102 may further comprise an inner layer 404 on inner surface 122 of tendon holding member 102. In an exemplary embodiment, inner layer 404 may comprise hyaluronic acid. In an exemplary embodiment, inner layer 404 may be configured to prevent adhesion of tendon 202 to inner surface 122 of tendon holding member 102 and. In an exemplary embodiment the hyaluronic acid present in inner layer 404 may aid in prevent adhesion of tendon 202 to inner surface 122 of tendon holding member 102. In an exemplary embodiment, when tendon 202 is not adhered to inner surface 122 of tendon holding member 102, tendon 202 may move and slide more easily inside tendon holding member 102. In an exemplary embodiment, a thickness of inner layer 404 may be in a range between 5 μm and 50 μm. In an exemplary embodiment, the thickness of inner layer 402 may be 12 μm. In an exemplary embodiment, inner layer 402 may be coated onto inner surface 122 of tendon holding member 102.

In an exemplary embodiment, prosthesis 100 may be utilized to replace any of annular pulleys in a human hand (A1-A5). In an exemplary embodiment, utilizing prosthesis 100 for compensating a damaged pulley system in a human hand may provide significant benefits. For example, a surgeon may fit prosthesis 100 into a hand of a person through a relatively simple and cost-effective single-step surgery procedure. Furthermore, the rehabilitation duration after the surgery for this person may be relatively short due to the simplicity of the surgery compared to typical surgeries.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Ends 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective spaces of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A prosthesis to replace a flexor tendon pulley of a human hand, the prosthesis comprising:
a tendon holding member with a hollow cylindrical shape, the tendon holding member comprising polyurethane, polycaprolactone, or a combination thereof, an inner diameter of an inner surface of the tendon holding member being 8 mm, a length of the tendon holding member being 10 mm, a thickness of the tendon holding member being 200 μm, the tendon holding member configured to receive and hold a tendon of the human hand, the tendon of the human hand associated with the flexor tendon pulley of the human hand, the tendon holding member comprising:
an outer layer on an outer surface of the tendon holding member, a thickness of the outer layer being 12 μm, the outer layer comprising hydroxyapatite, and
an inner layer on an inner surface of the tendon holding member, a thickness of the inner layer being 12 μm, the inner layer comprising hyaluronic acid;
a connecting plate with a rectangular shape attached to the tendon holding member, a length of the connecting plate being 15 mm, a width of the connecting plate being 9 mm, a thickness of the connecting plate being 1 mm, the length of the connecting plate corresponding to the length of the tendon holding member, the width of the connecting plate corresponding to the inner diameter of the inner surface of the tendon holding member, the connecting plate comprising titanium, the connecting plate configured to be attached to a phalange of the human hand, the phalange of the human hand associated with the tendon of the human hand, the connecting plate comprising a plurality of connecting holes, a connecting hole from the plurality of connecting holes configured to receive a connecting screw, the connecting screw configured to be screwed into the phalange of the human hand and, to thereby, secure the connecting plate to the phalange of the human hand, the plurality of connecting holes comprising:
a first connecting hole at a first corner of the connecting plate;
a second connecting hole at a second corner of the connecting plate;
a third connecting hole at a third corner of the connecting plate;
a fourth connecting hole at a fourth corner of the connecting plate; and
a fifth connecting hole at a center of the connecting plate, and
wherein the tendon holding member comprises an access hole on a top section of the tendon holding member, the access hole aligned with the fifth connecting hole, the access hole configured to provide access to the fifth connecting hole.

2. A prosthesis to replace a tendon sheath of a human body, the prosthesis comprising:
a tendon holding member with a hollow cylindrical shape, the tendon holding member configured to receive and hold a tendon of the human body, the tendon of the human body associated with the tendon sheath of the human body, and
a connecting plate attached to the tendon holding member, the connecting plate configured to be attached to a bone of the human body, the bone of the human body associated with the tendon of the human body.

3. The prosthesis of claim 2, wherein the tendon holding member comprises an outer layer on an outer surface of the tendon holding member, the outer layer comprising hydroxyapatite.

4. The prosthesis of claim 3, wherein the tendon holding element comprises an inner layer on an inner surface of the tendon holding member, the inner layer comprising hyaluronic acid.

5. The prosthesis of claim 4, wherein the connecting plate has a rectangular shape with a plurality of connecting holes, a connecting hole from the plurality of connecting holes configured to receive a connecting screw, the connecting screw configured to be screwed into the bone of the human body and, to thereby, secure the connecting plate to the bone of the human body.

6. The prosthesis of claim 5, wherein the plurality of connecting holes comprises:
a first connecting hole at a first corner of the connecting plate;
a second connecting hole at a second corner of the connecting plate;
a third connecting hole at a third corner of the connecting plate;
a fourth connecting hole at a fourth corner of the connecting plate; and
a fifth connecting hole at a center of the connecting plate.

7. The prosthesis of claim 6, wherein the tendon holding member comprises an access hole on a top section of the tendon holding member, the access hole aligned with the fifth connecting hole, the access hole configured to provide access to the fifth connecting hole.

8. The prosthesis of claim 7, wherein:
the tendon holding member comprises polyurethane, polycaprolactone, or a combination thereof, and
the connecting plate comprises titanium.

9. The prosthesis of claim 8, wherein:
an inner diameter of an inner surface of the tendon holding member is in a range between 4 mm and 12 mm;
a length of the tendon holding member is in a range between 5 mm and 15 mm; and
a thickness of the tendon holding member is in a range between 10 μm and 300 μm.

10. The prosthesis of claim 9, wherein:
a thickness of the outer layer is in a range between 5 μm and 50 μm; and
a thickness of the inner layer is in a range between 5 μm and 50 μm;
a length of the connecting plate is in a range between 7 mm and 20 mm, the length of the connecting plate corresponding to the length of the tendon holding member;
a width of the connecting plate is in a range between 4 mm and 12 mm, the width of the connecting plate corresponding to the inner diameter of the inner surface of the tendon holding member; and
a thickness of the connecting plate is in a range between 0.5 mm and 2 mm.

11. The prosthesis of claim 10, wherein:
the tendon sheath of the human body comprises a tendon pulley of the human hand; and
the bone of the human body comprises a phalange of the human hand, the phalange of the human hand associated with the tendon pulley of the human hand.
